(19) United States
(12) Patent Application Publication
(10) Pub. No.: US 2008/0194923 A1
(43) Pub. Date: Aug. 14, 2008

(54) ELECTRONIC PATIENT MONITOR WITH INTEGRATED SHOCK RESISTANT PIEZOELECTRIC SPEAKER

(75) Inventor: Toby E. Smith, Broken Arrow, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(73) Assignee: BED-CHECK CORPORATION, Tulsa, OK (US)

(21) Appl. No.: 12/106,005

(22) Filed: Apr. 18, 2008

Related U.S. Application Data
(63) Continuation-in-part of application No. 10/619,700, filed on Jul. 14, 2003.
(60) Provisional application No. 60/477,668, filed on Jun. 11, 2003.

(51) Int. Cl. A61B 5/00

(52) U.S. Cl. 600/300

(57) ABSTRACT

Disclosed is piezoelectric bender for use as an acoustic generating device. The acoustic generating device includes a piezoelectric material, a metal diaphragm, an electric circuit and mounting devices. The metal diaphragm is affixed to the piezoelectric material and has a nodal fulcrum at the nodal ring. The electric circuit is connected to the piezoelectric material and electrically activates the piezoelectric material. The mounting devices are constructed of insulating material and are positioned at the top and bottom of the metal diaphragm. The mounting devices support the metal diaphragm at the nodal fulcrum with an adhesive to resist impact damage.

### RELATED APPLICATIONS

This application claims priority to U.S. Provisional Application Ser. No. 60/477,668 filed Jun. 11, 2003 and is a continuation-in-part of U.S. patent application Ser. No. 10/619,700, the disclosures of which are incorporated herein by reference.

### FIELD OF THE INVENTION

This invention relates generally to patient monitoring systems and more particularly concerns devices that use audible alarms to inform of a patient's condition.

### BACKGROUND

It is well known that the use of electronic devices to monitor a patient's status is a growing trend in healthcare settings. This trend can be attributed to any number of factors including the increased vigilance that can be obtained with electronic monitoring (e.g., electronic monitors never sleep or leave the patient's vicinity for a break), decreased staffing costs (e.g., one caregiver can cover multiple patients), etc.

As a specific example of a patient condition that is especially suitable for electronic monitoring, consider the use of electronic patient monitors to help reduce the risk of a patient fall. By way of general background, a fall places a patient at risk of various injuries including sprains, fractures, and broken bones—injuries which in some cases can be severe enough to eventually lead to a fatality. Of course, those most susceptible to injury (e.g., the elderly and post surgical patients) are often those in the poorest general health and least likely to recover quickly from their injuries. In addition to the obvious physiological consequences of injuries to patients in poor health, there are also a variety of adverse economic and legal consequences that include the actual cost of treating the victim and, in some cases, caretaker liability issues.

In the past, it has been commonplace to treat patients that are prone to falling by limiting their mobility through the use of restraints, the underlying theory being that if the patient is not free to move about, he or she will not be as likely to fall. However, research has shown that restraint-based patient treatment strategies are often more harmful than beneficial and should generally be avoided—the emphasis today being on the promotion of mobility rather than immobility. Among the more successful mobility-based strategies for fall prevention include interventions to improve patient strength and functional status, reduction of environmental hazards, and staff training and identification and monitoring of high-risk hospital patients and nursing home residents.

Of course, direct monitoring high-risk patients, as effective as that care strategy might appear to be in theory, suffers from the obvious practical disadvantage of requiring additional staff if the monitoring is to be in the form of direct observation. Thus, the trend in patient monitoring has been toward the use of electrical devices to signal changes in a patient's circumstance to a caregiver who might be located either nearby or remotely at a central monitoring facility, such as a nurses' station. The obvious advantage of an electronic monitoring arrangement is that it frees the caregiver to pursue other tasks away from the patient. Additionally, when the monitoring is done at a central facility a single nurse can monitor multiple patients which can result in decreased staffing requirements.

Generally speaking, electronic monitors work by first sensing an initial status of a patient, and then generating a signal when that status changes, e.g., he or she has sat up in bed, left the bed, risen from a chair, etc., any of which situations could pose a potential cause for concern in the case of an at-risk patient. Electronic bed and chair exit monitors typically use a pressure sensitive switch in combination with a separate monitor/microprocessor. In a common exit monitor arrangement, a patient's weight resting on a pressure sensitive mat (i.e., a “sensing” mat) completes an electrical circuit, thereby signaling the presence of the patient to the microprocessor. When the weight is removed from the pressure sensitive switch, the electrical circuit is interrupted, which fact is sensed by the microprocessor. The software logic that drives the monitor is typically programmed to respond to the now-opened circuit by triggering some sort of alarm—either electronically (e.g., to the nursing station via a conventional nurse call system) or audibly (via a built-in audio alarm).

General information relating to mat sensors and electronic monitors for use in patient monitoring may be found in U.S. Pat. Nos. 4,179,692, 4,295,133, 4,700,180, 5,600,108, 5,633,627, 5,640,145, 5,654,694, and 6,111,509 (which concerns electronic monitors generally), and 7,079,795 (which concerns using pulse width modulation to control an alarm volume). Additional information may be found in U.S. Pat. Nos. 4,484,043, 4,565,910, 5,554,835, 5,623,760, 6,417,777, 7,078,676 (sensor patents), U.S. Pat. No. 7,030,764 pertaining to monitor and method for reducing the risk of decubitus ulcers, and U.S. Pat. No. 5,065,727 (holsters for electronic monitors), the disclosures of all of which patents are all incorporated herein by reference. Further, U.S. Pat. No. 6,307,476 (discussing a sensing device which contains a validation circuit incorporated therein), and U.S. Pat. Nos. 6,544,200 (for automatically configured electronic monitor alarm parameters), 6,696,653 and 6,858,811 (for a binary switch and a method of its manufacture), 6,864,795 (for a lighted splash guard), 7,079,036 (for alarm volume control using pulse width modulation), 6,897,781 (for an electronic patient monitor and white noise source for soothing a patient to sleep after they have turned), and U.S. patent application Ser. No. 11/507,418 (for a method and apparatus for temporarily disabling a patient monitor) are similarly incorporated herein by reference.

Note that the instant invention is suitable for use with a wide variety of patient sensors in addition to pressure sensing switches including, without limitation, temperature sensors, patient activity sensors, cardiac sensors, toilet seat sensors (see, e.g., U.S. Pat. No. 5,945,914), wetness sensors (e.g., U.S. Pat. No. 6,292,102), bed pressure sore sensors (e.g., U.S. Pat. Nos. 6,646,556, 6,987,232, and 7,078,676), thermal sensors (U.S. patent application Ser. No. 11/132,772), etc. Thus, in the text that follows the terms “mat” or “patient sensor” should be interpreted in its broadest sense to apply to any sort of patient monitoring sensor or device, whether the sensor is pressure sensitive or not.

A key component of a patient monitor is its loudspeaker. Since in many cases situations caregivers rely exclusively on the audible alert provided by such monitors, it is important that this component be reliable and resistant to the sort of damage—both accidental and intentional—that is often encountered in the field. In more particular, since monitors can easily be dropped onto a hard surface or accidentally struck, it is important that the loudspeaker be able to withstand lateral and other such shocks and to continue to broadcast at near-full volume. However, heretofore that has often not been the case.

Piezoelectric transducers have been used in patient monitoring applications as speakers for purposes of creating alarms and other sounds in response to electric signals that originate from a microprocessor or other CPU-type device within the monitor. Piezoelectric materials are used in sound generating applications due to low power consumption requirements, small space requirements, and readily available materials. These design advantages have led to use in buzzers or other types of small sound generating units for portable and easily moveable systems.

Those of ordinary skill in the art will recognize that these types of sound generating devices (i.e., “speakers” generically, hereinafter) typically include some combination of a piezoelectric material, a thin metal diaphragm, an electrical circuit and a mounting device. The piezoelectric material and the metal diaphragm are usually bonded together and connected to the electrical circuit. Electrical activation of the piezoelectric material causes it to alternately expand and contract, thereby translating electrical energy to mechanical energy. This movement of the piezoelectric material in turn bends the metal diaphragm to which it is bonded (hence the term “bender”), causing an acoustic wavefront that generates sound. The mounting device holds the piezoelectric material and the metal diaphragm (collectively the “bender”) in proper orientation to allow vibration of the bender while avoiding contact between the bender and other structures that may impede or attenuate the vibration.

One prior art method of mounting the bender is to affix a mounting ring to a single side of the bender. However, using a single mounting ring leaves the acoustic generating device susceptible to mechanical shock that can dislodge the bender from the mount, thereby causing a malfunction of the output. Also, the mount has a tendency to attenuate the sound generated by the bender and absorb some of the acoustic energy into the mounting adhesive, thereby decreasing the decibel level of the acoustic output.

Other prior art methods attempt to mount the bender on both the front and back surfaces using electrical conductors that provide electrical input. A bender mounted in this fashion is quite susceptible to lateral shock that can dislodge the bender from proper positioning on the mount and cause mechanical failure. This method of mounting can also attenuate the sound below a desired magnitude required for a useful audible level due to a clamping action from the front and back mounts, thereby reducing the bender flexibility.

Other prior art methods have utilized, for example, purely mechanical mounts. However, such bender mounting methods have proven to be highly susceptible to lateral shock which tends to make them unsuitable for in-field medical applications. Others have sought to hold the bender in place within its case using adhesive, but prior art devices using this approach have suffered a decrease output sound level or intensity as a consequence of this approach.

There is therefore a continued need for improving the capabilities of piezoelectric speakers for use in patient monitoring devices. It is to these and other deficiencies in the prior art that the present invention is directed.

### SUMMARY OF THE INVENTION

Preferred embodiments of the present invention provide a speaker for use in a patient monitoring device that includes a piezoelectric material, a metal diaphragm, an electric circuit and mounting devices. The metal diaphragm is bonded to the piezoelectric material and has a nodal fulcrum or a nodal ring, as most piezoelectric materials and diaphragms (e.g., benders) are of circular and concentric construction. The electric circuit is connected to the piezoelectric material and electrically activates the piezoelectric material. The mounting devices are preferably constructed of insulating material and are positioned at the top and bottom of the metal diaphragm. The mounting devices support the metal diaphragm along the nodal fulcrum (e.g., nodal ring in a round bender) with an adhesive.

The foregoing has outlined in broad terms the more important features of the invention disclosed herein so that the detailed description that follows may be more clearly understood, and so that the contribution of the instant inventor to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Further, the disclosure that follows is intended to apply to all alternatives, modifications and equivalents as may be included within the spirit and scope of the invention as defined by the appended claims. Finally, it should be understood that the phraseology and terminology employed herein are for the purpose of description and should not be regarded as limiting, unless the specification specifically so limits the invention.

While the instant invention will be described in connection with one or more preferred embodiments, it will be understood that it is not intended to limit the invention to those embodiments. On the contrary, it is intended to cover all alternatives, modifications and equivalents as may be included within the spirit and scope of the invention as defined by the appended claims.

### BRIEF DESCRIPTION OF THE DRAWINGS

Other objects and advantages of the invention will become apparent upon reading the following detailed description and upon reference to the drawings in which:

FIG. 1 is an elevational cross-sectional view of a piezoelectric bender.

FIG. 2 is an elevational view of the vibrational mode of the bender of FIG. 1.

FIG. 3 is a bottom view of a standard circular bender.

FIG. 4 is a circuit diagram of a driver for use with the bender of FIG. 1.

FIG. 5 is a cross-sectional view of an acoustic generating device constructed in accordance with a preferred embodiment of the present invention.

FIG. 6 illustrates a typical environment of the invention as it might be used on a hospital bed.

FIG. 7 contains a schematic illustration of a preferred embodiment of the invention as it might appear in use on a wheel chair.

FIG. 8 contains a representation of a preferred monitor embodiment.

FIG. 9 contains a simple schematic illustration of a preferred monitor electrical arrangement.

### DETAILED DESCRIPTION OF THE PREFERRED EMBODIMENT

### General Environment of the Invention

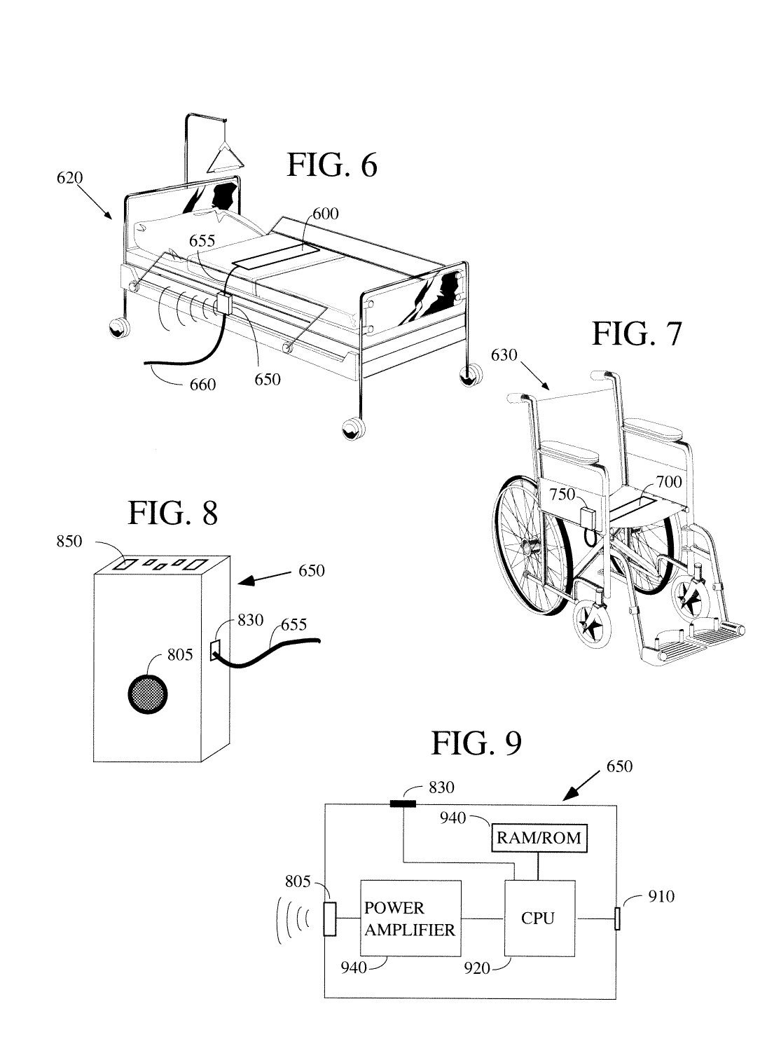

Turning first to FIG. 6 wherein the general environment of one specific embodiment of the instant invention is illustrated, in a typical bed (or chair) exit arrangement a pressure sensitive mat 600 sensor is placed on a hospital bed 620 where it will lie beneath a weight-bearing portion of the reclining patient's body, usually the buttocks and/or shoulders. Generally speaking, the mat 600/electronic monitor 650 combination works as follows. When a patient is placed atop the mat 600, pressure generated by the patient's weight compresses it, thereby closing an internal electrical circuit. This circuit closure is sensed by the associated electronic patient monitor 650 and, depending on its design, this circuit closure may signal the monitor 650 to begin monitoring the patient via the sensing mat 600. Additionally, in some embodiments, the monitoring phase is initiated manually by the caregiver using a switch on the exterior of the monitor 650 that has been provided for that purpose.

After the monitoring function is engaged, if the patient attempts to leave the support surface, the sensing mat 600 detects the change in the patient's condition, thereby changing its internal circuitry causing a variation which is sensed by the attached electronic patient monitor 650. The patient monitor 650, which conventionally contains a microprocessor therein, then signals the caregiver per its pre-programmed instructions. In some cases, the signal will amount to an audible alarm or siren that is emitted from the unit 50. In other cases, an electronic signal could also be sent to a remote nurses/caregivers station wirelessly or via electronic communications line 60. In still another preferred arrangement, the patient monitor 50 will sound an audio alarm locally and simultaneously send the alarm signal to the nurses station. Note that additional electronic connections not pictured in this figure might include a monitor power cord to provide a source of AC power although, as generally pictured in this figure, the monitor 50 can certainly be configured to be either battery (to include capacitive storage) or AC powered, although a battery or other mobile power source is generally preferred in the case of a monitor that is attached to a wheelchair.

In another common arrangement, and as is illustrated in FIG. 7, a pressure sensitive chair sensor 700 might be placed in the seat of a wheel chair or the like for purposes of monitoring a patient seated therein. As has been described previously, a typical configuration utilizes a pressure sensitive mat 700 which is connected to electronic chair monitor 750 that is attached to the chair 630. Because it is anticipated that the patient so monitored might want to be at least somewhat mobile, the monitor 750 will usually be battery powered and will often signal a chair-exit event via an integral speaker (or, e.g., via a wireless link), rather than via a hardwired nurse-call interface.

Broadly speaking, the electronic patient monitors that are referred to herein work by first sensing an initial status of a patient, and then generating a signal when that status changes (e.g., the patient changes position from laying or sitting to standing, the sensor changes from dry to wet, exhibits a fever, etc.). FIGS. 8 and 9 illustrate some details of such a monitor. In a typical configuration, a top panel 850 will contain some combination of switches and indicators (e.g., LCD readouts, signal lights, etc.) that can be used to control the operations of the monitor 650. Speaker 805, and preferably a speaker made according to the instant invention, is used to give audible alarms, feedback, etc. Connector 830 provides a means by which the CPU 920 can transmit an alert to, for example, a remote nurses station.

Preferably the monitor will utilize connector 910 (FIG. 9) to interface with the patient sensor 600. In some preferred configurations the interface 910 is compatible with an RJ-11-type jack. Preferably the sensor will be a mat-type pressure sensitive sensor, however it should be clear that the type of sensor—and its means of electrical attachment—is immaterial to the operation of the instant invention. That is, no matter what form the attached sensor might take (e.g., presence/absence, position, wetness, temperature, pressure, movement, etc.), the mode of operation of the instant patient monitor would generally be the same, i.e., detect a change in a patient condition and issue a signal in response. According to the preferred arrangement, each monitor unit is equipped with a speaker 805 according to the instant invention, through which an audio alarm may be issued.

Turning now to FIG. 9 wherein a schematic diagram of a preferred embodiment 650 is presented, the CPU 920 will have access to some amount of storage 940 which could be used to store its controlling program. Preferably, the storage 940 will take the form of non-volatile memory (e.g., ROM, flash RAM, etc.), which might be either internal or external to the CPU 920. That being said, those of ordinary skill in the art will recognize that conventional computer memory is only one of many possible storage sources that might be used and alternatives such as magnetic disk, remote hard disk (e.g., booting over a network), optical disk, magneto-optical disk, etc. Thus, for purposes of the instant invention, when the words “memory” or “storage” are used, those terms should be interpreted in the broadest sense to include any sort of electronic data storage that is accessible by the CPU 920, whether that storage is internal to the monitor 300 or external to it.

In electronic communication with CPU 920, and preferably external to it, is a an optional power amplifier 940, the purpose of which is to amplify the signal that is sourced in CPU 920. The speaker 310 is a piezoelectric device as is discussed more fully below and, especially preferably, it will be a piezoelectric device that is driven directly from the microprocessor without an intervening amplifier.

Preferably, the CPU 940 will control the reading of the front panel 850 switches and display of control information according to methods well known to those of ordinary skill in the art.

### Preferred Embodiments

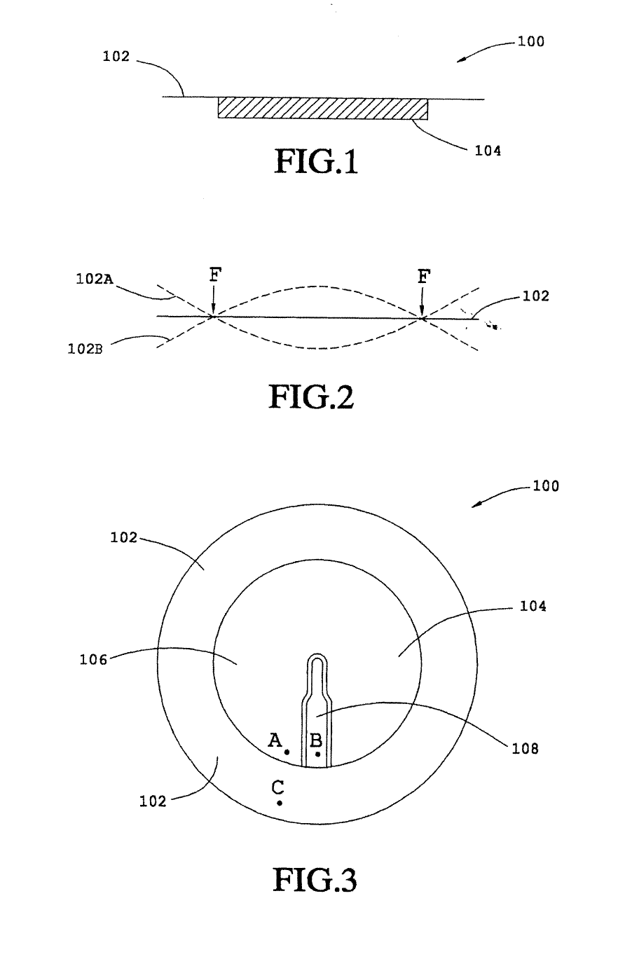

Turning now to a discussion of the instant inventive shock resistant speaker, in accordance with a preferred embodiment of the present invention, FIG. 1 shows an elevational view of a bender 100 for use as an acoustic generating device. A metal diaphragm 102, such as a thin metal plate, is attached to piezoelectric material 104 as known in the art. The metal diaphragm 102 is preferably constructed from brass, stainless steel, or other suitable material. Likewise, the piezoelectric material 104 is constructed of a piezoelectric ceramic (such as lead-zirconate-titanate), but other materials that have piezoelectric properties are also suitable for use as an acoustic generating device. In addition to the embodiments disclosed herein, it is also assumed that the bender 100 could be constructed in a variety of shapes to match specific applications.

Referring now to FIG. 2, the metal diaphragm 102 is shown in a sound producing vibrational mode. When the piezoelectric material 104 (not shown in FIG. 2) is electrically activated, the metal diaphragm 102 enters the vibrational mode as indicated by the deflected metal diaphragm 102A, 102B. This alternating bending motion of the metal diaphragm 102 produces sound waves that generate the desired sound from the acoustic generating device. Also indicated in FIG. 2 are two nodal fulcrums (designated by F) that mark the points of minimal deflection for the metal diaphragm 102. Although in cross section the two nodal fulcrums F appear as distinct points, those of ordinary skill in the art will recognize that in reality in this case (i.e., with a circular bender) each is actually a point on a nodal ring which exhibits a minimum deflection property throughout its circumference (i.e., the nodal fulcrums in the case of a round piezoelectric material/bender combination will comprise a circular curve).

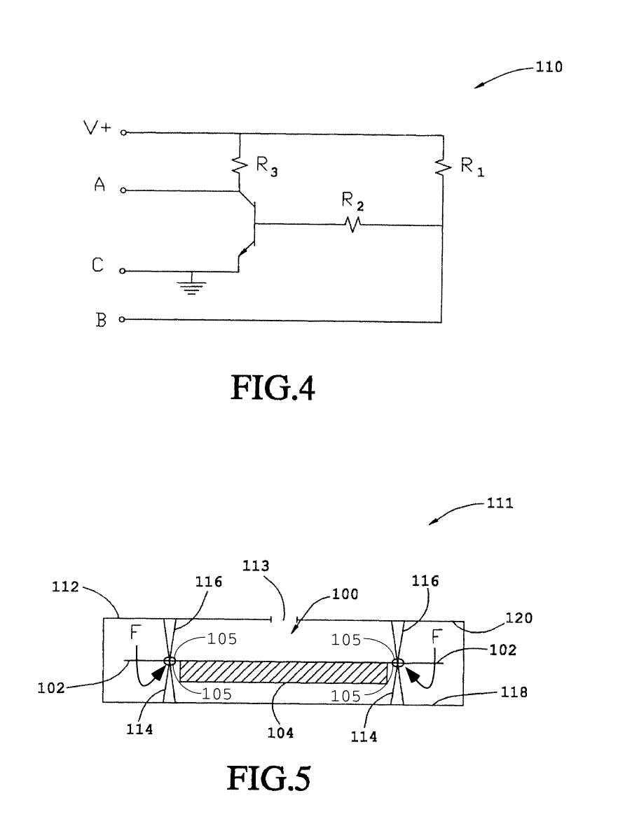

FIG. 3 is a bottom view of the bender 100 of FIG. 1. The bender 100 preferably includes electrical connectivity points A, B and C for use in electrically activating the bender 100 in accordance with techniques known in the art. The piezoelectric material 104 includes an input electrode 106 and feedback electrode 108, which are connected to electrical connectivity points A and B. The metal diaphragm 102 is connected to connectivity point C, as demonstrated further by FIG. 4.

Shown in FIG. 4 is an electric circuit 110 that provides an internal drive circuit for the bender 100. Nodes A, B and C correspond to electrical connectivity points A, B and C in the bender 100, and are typically soldered directly to those points to provide a reliable connection. Although the electric circuit 110 demonstrates a common circuit for the activation of a piezoelectric material, the present invention is not so limited. Many other configurations are equally useful, such as push-pull circuits, and circuits that employ inverters, capacitors and inductors, any of which can provide the necessary excitation in the bender assembly 100 necessary to produce sound. The present invention can also be directly driven by logic level outputs from direct logic circuits or from a microprocessor, with or without the use of feedback from “B” 108.

Referring now to FIG. 5, shown therein is an acoustic generating device 111. The acoustic generating device 111 preferably includes a housing 112; the bender 100; the electric circuit 110 (not shown in FIG. 5); and mounting rings 114, 116. In a preferred embodiment, the housing 112 includes an aperture 113 that releases the sound produced by the bender 100.

Lower mounting ring 114 is provided below the bender 100 and is secured to the bottom side of the bender 100 using adhesive 105 (such as glue, epoxy or other suitable adhesive) at the nodal fulcrums (which comprise a ring with a round bender), designated as “F.” It is preferable to secure the lower mounting ring 114 to the base 118 of the device 111 by molding the lower mounting ring into the housing 112 or by applying adhesive between the lower mounting ring 114 and the base 118. The lower mounting ring 114 is preferably constructed of plastic or other suitable insulator.

Similarly, upper mounting ring 116 is provided above the bender 100 and is secured to the top side of the bender 100 using adhesive 105 at the nodal fulcrums, designated as “F.” Preferably the upper mounting ring 116 is secured to the top 120 of the housing 112 by using adhesive between the upper mounting ring 116 and the top 120 or by molding the upper mounting ring 116 into the housing 112. The upper mounting ring 116 is preferably constructed of plastic or other suitable insulator.

The nodal fulcrums are useful points by which the bender 100 can be mounted. Mounting at these points is beneficial for acoustic generating devices due to the limited attenuation that occurs, thereby allowing the device to function acoustically at an optimal level. Mounting at other points along the bender 100 tends to damp the vibration, thereby decreasing the sound generation capability of the device and, as a consequence, should generally be avoided.

Placement of the upper and lower mounting rings 116, 114 as described above prevents vertical movement of the bender 100 that can dislodge the bender 100 from an opposing mounting ring. Also, the use of adhesive 105 to attach either one or both of the mounting rings 114, 116 to the bender 100 prevents lateral movement that can cause the bender 100 to become dislodged or to be moved from a position of support at the nodal fulcrum F. Maintaining the support of the bender 100 at the nodal fulcrum F prevents attenuation of the generated sound. It will be understood that the shape of the bender 100 dictates the shape of the mounting rings 114, 116 in that changing the shape of the bender 100 will also tend to change the shape of the nodal ring itself.

Although the nodal fulcrum in FIG. 5 is shown to be on the metal diaphragm 102 at a location beyond the piezoelectric material 104, it is envisioned that the nodal fulcrum can occur at various points along the metal diaphragm 102 depending on the design of the bender assembly itself, and the exact shape or location of the nodal fulcrum in a particular instance is not important in the practice of the instant invention. For example, the nodal fulcrum could be at the edges of the piezoelectric material 104, or even inside the outer radius of the piezoelectric material 104. In either case, the appropriate geometric mounting devices should support the bender 100 along the nodal curve, wherever that curve might be found in the specific bender that is used. Of course, if the diaphragm 102 and piezoelectric material 104 are both round, in the preferred embodiment the mounting devices will likely similarly be round in cross section so as to support the speaker 100 along the circular nodal fulcrum.

Additionally, it should be noted that neither the bender nor its piezoelectric material need be circular. In fact, the cross section of FIG. 5 also is applicable to a rectangular bender/piezoelectric material combination, although the nodal ring (or nodal fulcrums) in this instance would comprise two straight lines.

In accordance with one aspect of a preferred embodiment, the present invention provides an apparatus for protecting a bender assembly, thereby increasing the shock resistance and operating life of the acoustic generating device. It is to be understood that even though numerous characteristics and advantages of various embodiments of the present invention have been set forth in the foregoing description, together with details of the structure and functions of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. It will be appreciated by those skilled in the art that the teachings of the present invention can be applied to other systems without departing from the scope and spirit of the present invention.

Note that if a microprocessor 920 is utilized as a component of the monitor 650 or 750, the only requirement that such a component must satisfy is that it must minimally be an active device, i.e., one that is programmable in some sense, that it is capable of recognizing signals from a bed mat or similar patient sensing device, and that it is capable of initiating the sounding of one or more alarm sounds in response thereto. Of course, these sorts of modest requirements may be satisfied by any number of programmable logic devices (“PLD”) including, without limitation, gate arrays, FPGA's (i.e., field programmable gate arrays), CPLD's (i.e., complex PLD's), EPLD's (i.e., erasable PLD's), SPLD's (i.e., simple PLD's), PAL's (programmable array logic), FPLA's (i.e., field programmable logic array), FPLS (i.e., fuse programmable logic sequencers), GAL (i.e., generic array logic), PLA (i.e., programmable logic array), FPAA (i.e., field programmable analog array), PsoC (i.e., programmable system-on-chip), SoC (i.e., system-on-chip), CsoC (i.e., configurable system-on-chip), ASIC (i.e., application specific integrated chip), etc., as those acronyms and their associated devices are known and used in the art. Further, those of ordinary skill in the art will recognize that many of these sorts of devices contain microprocessors integral thereto. Thus, for purposes of the instant disclosure the terms “processor,” “microprocessor” and “CPU” (i.e., central processing unit) should be interpreted to take the broadest possible meaning herein, and such meaning is intended to include any PLD or other programmable device of the general sort described above.

Additionally, in those embodiments taught herein that utilize a clock or timer or similar timing circuitry, those of ordinary skill in the art will understand that such functionality might be provided through the use of a separate dedicated clock circuit or implemented in software within the microprocessor. Thus, when “clock” or “time circuit” is used herein, it should be used in its broadest sense to include both software and hardware timer implementations.

Note further that a preferred electronic monitor of the instant invention utilizes a microprocessor with programming instructions stored therein for execution thereby, which programming instructions define the monitor's response to the patient and environmental sensors. Although ROM is the preferred apparatus for storing such instructions, static or dynamic RAM, flash RAM, EPROM, PROM, EEPROM, or any similar volatile or nonvolatile computer memory could be used. Further, it is not absolutely essential that the software be permanently resident within the monitor, although that is certainly preferred. It is possible that the operating software could be stored, by way of example, on a floppy disk, a magnetic disk, a magnetic tape, a magneto-optical disk, an optical disk, a CD-ROM, flash RAM card, a ROM card, a DVD disk, or loaded into the monitor over a network as needed. Additionally, those of ordinary skill in the art will recognize that the memory might be either internal to the microprocessor, or external to it, or some combination. Thus, RAM/ROM or “program memory” as that term is used herein should be interpreted in its broadest sense to include the variations listed above, as well as other variations that are well known to those of ordinary skill in the art.

Finally, it should be noted that, although the preferred embodiment calls for the mounting devices 114 and 116 to be non-conductive, that is not strictly required. It should be recognized by those of ordinary skill in the art that conductive mounting devices could be used to serve as electrical contact “C” in FIG. 3 if it were so desired.

Thus, it is apparent that there has been provided, in accordance with the invention, a shock-resistant speaker that fully satisfies the objects, aims and advantages set forth above. While the invention has been described in conjunction with specific embodiments thereof, it is evident that many alternatives, modifications and variations will be apparent to those skilled in the art and in light of the foregoing description. Accordingly, it is intended to embrace all such alternatives, modifications and variations as fall within the spirit of the appended claims.

What is claimed is:

1. A shock resistant electronic patient monitor, comprising:
(a) a sensor positionable to be proximate to the patient, said sensor at least for monitoring a changeable status of a patient;
(b) a CPU in electronic communication with said sensor, said CPU being at least for monitoring said changeable status of the patient and initiating an alarm in response thereto;
(c) computer storage in electronic communication with said CPU, said computer storage containing therein at least a plurality of computer instructions executable by said CPU for monitoring the status of the patient and sounding said alarm in response thereto:
(d) a shock resistant speaker in electronic communication with said CPU, said speaker at least for sounding said alarm, said speaker comprising:
(d1) a piezoelectric material,
(d2) a metal diaphragm bonded to said piezoelectric material, said metal diaphragm and piezoelectric material together having a nodal fulcrum,
(d3) an upper mounting device positioned on an upper surface of said metal diaphragm, said upper mounting device supporting said diaphragm along said nodal fulcrum and being held against said nodal fulcrum by an adhesive, and,
(d4) a lower mounting device positioned on a lower surface of said metal diaphragm, said lower mounting device supporting said diaphragm along said nodal fulcrum and being held against said nodal fulcrum by said adhesive, wherein said metal diaphragm and said piezoelectric material are solely supported by said upper and lower mounting devices.

2. The shock resistant electronic patient monitor of claim 1, wherein the metal diaphragm is substantially round and wherein said nodal fulcrum is circular.

3. The shock resistant electronic patient monitor of claim 1, wherein said upper and lower mounting devices are attached to the inside of a housing that at least partially encloses said metal diaphragm.

4. The shock resistant electronic patient monitor of claim 1, wherein said shock resistant speaker further comprises a power amplifier in electronic communication with said CPU and with said shock resistant speaker, said power amplifier being at least for amplifying signals alarm signals said CPU.

5. The shock resistant electronic patient monitor of claim 1, wherein one of said mounting devices is attached to said piezoelectric material with said adhesive.

6. The shock resistant electronic patient monitor of claim 1, wherein at least one of the mounting devices is attached to said metal diaphragm with said adhesive.

7. The shock resistant electronic patient monitor of claim 1, wherein said upper mounting device continuously supports said diaphragm along an entirety of said nodal fulcrum and said mounting device is held against said entirety of said nodal fulcrum by an adhesive.

8. The shock resistant electronic patient monitor of claim 7, wherein said nodal fulcrum is substantially circular.

9. The shock resistant electronic patient monitor of claim 1, wherein said upper mounting device and said lower mounting device are both made of a same insulating material.

10. The shock resistant electronic patient monitor of claim 1, wherein said piezoelectric material is ceramic.

11. A shock resistant electronic patient monitor for use in monitoring a changeable status of a patient, wherein is provided a patient sensor at least for monitoring the changeable status of the patient, comprising:
(a) a CPU in electronic communication with said sensor, said CPU being programmable to monitor the changeable status of the patient and to initiate an alarm in response thereto;
(b) a shock resistant speaker in electronic communication with said CPU, said speaker at least for sounding said alarm, said speaker comprising:
(b1) a piezoelectric material,
(b2) a metal diaphragm bonded to said piezoelectric material, said metal diaphragm and piezoelectric material together having a nodal fulcrum,
(b3) an upper mounting device constructed of insulating material and positioned on an upper surface of said metal diaphragm, said upper mounting device supporting said diaphragm along said nodal fulcrum and being held against said nodal fulcrum by an adhesive,
(b4) a lower mounting device constructed of insulating material and positioned on a lower surface of said metal diaphragm, said lower mounting device supporting said diaphragm along said nodal fulcrum and being held against said nodal fulcrum by said adhesive, wherein said metal diaphragm and said piezoelectric material are solely supported by said upper and lower mounting devices, and,
(b5) a housing that substantially encloses said piezoelectric material, said metal diaphragm, and said upper and lower mounting devices.

12. The shock resistant electronic patient monitor of claim 11, wherein said metal diaphragm is substantially round.

13. The shock resistant electronic patient monitor of claim 11, wherein the mounting devices are attached to the inside of the housing.

14. The shock resistant electronic patient monitor of claim 11, wherein one of said mounting devices is attached to said piezoelectric device with the adhesive.

15. The shock resistant electronic patient monitor of claim 11, wherein at least one of the mounting devices is attached to the metal diaphragm with the adhesive.

16. The shock resistant electronic patient monitor of claim 11, wherein at least one of said upper and lower mounting devices is substantially round in cross section.

17. The shock resistant electronic patient monitor of claim 1, wherein said upper mounting device and said lower mounting device are both made of a same insulating material.

18. The shock resistant electronic patient monitor of claim 11, wherein said nodal fulcrum is a nodal ring.

19. The shock resistant electronic patient monitor of claim 11, wherein the piezoelectric material is ceramic.

20. A shock resistant electronic patient monitor for use in monitoring a changeable status of a patient, wherein is provided a patient sensor at least for monitoring the changeable status of the patient, comprising:
(a) a CPU in electronic communication with said sensor, said CPU being programmable to monitor the changeable status of the patient and to initiate an alarm in response thereto;
(b) a shock resistant speaker in electronic communication with said CPU, said speaker at least for sounding said alarm, said speaker comprising:
(b1) a bender, said bender having a nodal fulcrum,
(b2) an upper mounting device positioned on an upper surface of said bender, said upper mounting device supporting said bender along said nodal fulcrum and being held against said nodal fulcrum by an adhesive,
(b3) a lower mounting device positioned on a lower surface of said bender, said lower mounting device supporting said bender along said nodal fulcrum and being held against said nodal fulcrum by said adhesive, and,
(b4) a housing that substantially encloses at least said bender and said upper and lower mounting devices.
